(19) United States
(12) Patent Application Publication
(10) Pub. No.: US 2011/0040194 A1
(43) Pub. Date: Feb. 17, 2011

(54) METHOD AND SYSTEM FOR DETERMINING CARDIAC PARAMETERS

(76) Inventor: Rajendra Prasad Jadiyappa, Bangalore (IN)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/539,894

(22) Filed: Aug. 12, 2009

(51) Int. Cl. A61B 5/021

(52) U.S. Cl. 600/485

(57) ABSTRACT

The present invention explains a method and system for providing all the relevant cardiac parameters in real time and in a fast manner, which are required for the cardiac analysis. The invention involves determining a first area and a second area, the first area under a waveform representing an aortic pressure, and extends between an onset of the systole and the end of the systole and the second area extends between a waveform representing a left ventricle pressure and a waveform adapted to represent the left atrium pressure of the same systole. The method further involves presenting a functional relationship between the first area and the second area on the one hand side and the cardiac parameters on the other hand side and then finally determining the cardiac parameters based on the first area and the second area by using the representation of the functional relationship.

### FIELD OF INVENTION

The present invention relates to medical analysis, particularly a method and system for the estimation of volumetric cardiac parameters using pressure waveforms.

### BACKGROUND OF INVENTION

Cardiac volumetric analysis is used clinically to evaluate cardiac function in the diagnosis of cardiovascular disease, and thereby guide therapeutic decisions in complex clinical situations.

Cardiac output is defined as the volume of blood pumped by the heart per minute, which is an important measure of heart pumping capacity and blood circulation. Till now there exist various methods of estimation of cardiac output in cathlab such as thermo dilution, dye-dilution, fick principle, left ventricle angiography, etc. However each one of them have their own limitations and moreover it is not possible to obtain a continuous and faster estimation and display of cardiac output in real time with these methods. Also these methods cannot provide all the relevant cardiac volumetric parameters in real time, which are required for the cardiac analysis.

The commonly used method for estimating various volumes during cardiac catheterization is by left ventricular angiography, which involves injecting dye into the left ventricle and analyzing the frame of image during end diastole to obtain end diastolic volume and frame of image during end systole to obtain end systolic volume using software analytical tools. However this would involve few hazards associated with iodine contrast medium injection like dye allergy, hemodynamic instability, pulmonary congestion and renal impairment. Moreover the said method is also risky in cases of medical conditions like severe congestive cardiac failure and renal failure.

### SUMMARY OF INVENTION

In view of the foregoing, an embodiment herein includes a method of determining a cardiac parameter comprising: determining a first area under a waveform representing an aortic pressure, said first area extends between an onset of the occurrence of a systole and the end of the systole; providing a representation of a functional relationship between the first area and the cardiac parameter; and determining the cardiac parameter, based on the first area by using the representation of the functional relationship.

In another embodiment the said object is achieved by providing a method of determining a cardiac parameter comprising: determining a second area, said second area extends between a waveform representing a left ventricle pressure and a waveform adapted to represent the left atrium pressure in a systole; providing a representation of a functional relationship between the second area and the cardiac parameter; and determining the cardiac parameter, based on the second area by using the representation of the functional relationship.

Another embodiment includes a system for measuring cardiac parameters comprising: a determining module adapted to determine a first area and a second area, the first area under a waveform representing an aortic pressure, and extends between an onset of systole and the end of the systole and the second area extends between a waveform representing a left ventricle pressure and a waveform adapted to represent the left atrium pressure of the same systole; a representation module, adapted to present a functional relationship between the first area and the second area on the one hand side and the cardiac parameters on the other hand side; and a cardiac parameter determining module, for determining the cardiac parameters based on the first area and the second area by using the representation of the functional relationship.

### BRIEF DESCRIPTION OF THE DRAWINGS

The present invention is further described hereinafter with reference to illustrated embodiments shown in the accompanying drawings, in which:

FIG. 1 illustrates a graphical representation of the pressure waveforms for computing the areas under the curves for estimating the cardiac parameters,

FIG. 2 illustrates a database which provide the functional relationship between the area under the curve and the volume according to an embodiment of the invention,

FIG. 3 illustrates a system for measuring cardiac parameters according to an embodiment of the invention, and

FIG. 4 illustrates the pressure waveforms of aorta, left ventricle and pulmonary capillary wedge pressure obtained and displayed simultaneously on a common scale according to an embodiment of the invention.

### DETAILED DESCRIPTION OF INVENTION

Since the pressure waveforms are easily available in any recording system in cathlab, these waveforms can be used for the volumetric analysis of the heart, specifically for left ventricle to obtain various cardiac parameters. These pressure waveforms are basically invasive pressure waveforms. Hemodynamic assessment is commonly done during cardiac catheterization. This involves inserting catheters to gain access to various heart chambers, aorta or any associated vessel of heart and record pressure waveforms. The recording system in the cathlab during examination acquires the aortic pressure waveform, left ventricular pressure waveform, and a waveform adapted to represent the left atrium pressure obtained using a catheter insertion.

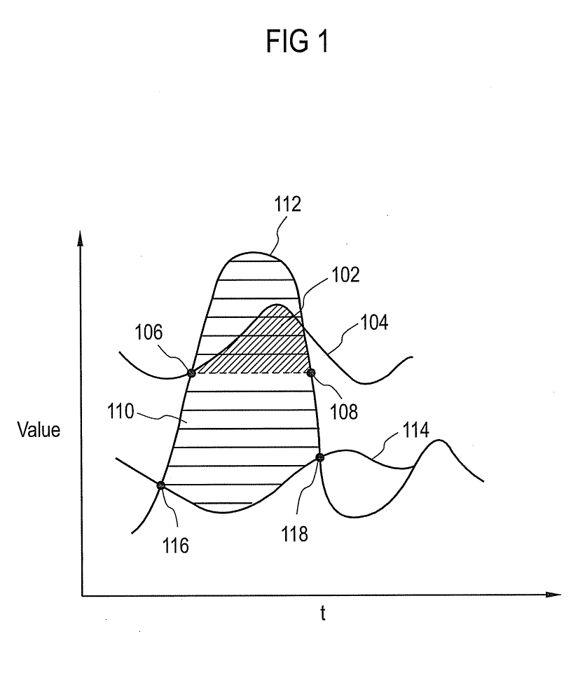

FIG. 1, shows the pressure waveforms of aorta, left ventricle and pulmonary capillary wedge pressure obtained and displayed simultaneously on a common scale in overlapping pattern for analysis. The x-axis represents the time scale and the y-axis represents the corresponding values. The method involves the step of determining a first area 102 under a waveform 104 representing an aortic pressure, said first area 102 extends between an onset of the occurrence of a systole of the heart and the end of the systole. Systole is basically the process of contraction of the heart, which results in pumping of blood.

The area under the curve during systole for the waveform 104 representing an aortic pressure is first estimated. For convenience of explanation, the waveforms in the invention are sometimes referred to as curves in different portions of the description. The intersection 106 of waveform 104 representing an aortic pressure and waveform 112 representing a left ventricle pressure during the onset of systole is identified since this marks the crossover of pressure rise in the left ventricle over the aortic pressure and would correspond to onset of ejection of blood from the left ventricle of the heart to the aorta. A horizontal line is drawn till it intersects the waveform 112 representing a left ventricle pressure towards the end of systole at a point 108. This convention is followed to determine the base of the waveform 104 representing an aortic pressure for calculating the first area under the wave form 104.

The method then involves, determining a second area 110, said second area 110 extends between a waveform 112 representing a left ventricle pressure and a waveform 114 adapted to represent the left atrium pressure in the same systole. The intersection 116, of the waveform 112 representing a left ventricle pressure and a waveform 114 adapted to represent the left atrium pressure towards onset of systole is identified. This marks the crossover of pressure rise in the left ventricle over the left atrium and would correspond to the end of left ventricle diastolic filling and hence the maximum volume of blood available in the left ventricle. The intersection 118, of the waveform 112 representing a left ventricle pressure and a waveform 114 adapted to represent the left atrium pressure towards the end of systole is then identified. This marks the crossover of pressure rise in the left atrium over left ventricle and would correspond to the onset of left ventricular filling and hence correspond to the minimum volume of blood available in the left ventricle. Thus the systolic waveform adapted to represent the left atrium pressure between the intersection 116 and the intersection 118 would form the base of the waveform 112 for determining the second area 110.

Conventionally, catheters are inserted through femoral arteries, under fluoroscopic guidance. One catheter is placed in the aorta and another catheter is placed in the left ventricle. The other ends of catheter are connected to an external pressure transducer and recording system to display and record pressure waveforms. Pulmonary capillary wedge pressure obtained would be a surrogate marker of left atrium pressure. For recording pulmonary capillary wedge pressure, another catheter is inserted through femoral vein to reach inferior venacava, then right atrium to right ventricle and finally through pulmonary artery to reach pulmonary capillary.

Alternatively in case of defect in the inter-atrial septum, the catheter can be directed from right atrium directly into the left atrium to obtain left atrium pressure. Alternatively if the heart rate is regular and significant beat to beat variations of the heart are absent, femoral arterial pressure or radial artery pressure obtained through the side arm of an inserted arterial sheath used for inserting the catheters, can be used instead of aortic pressure waveform after adjusting for time shift and pressure waveform can be overlapped over the left ventricle pressure waveform on a common scale for further analysis, thereby preventing insertion of catheter exclusively for recording aortic pressure. Thus the pressure waveforms of aorta, left ventricle and pulmonary capillary wedge pressure are obtained and displayed simultaneously on a common scale in overlapping pattern for analysis.

The areas under the waveforms represent cardiac parameters. The first area 102 represents a stroke volume (SV) and the second area 110 represents a left ventricle systolic volume (LVSV). Stroke volume is the amount of blood ejected by the left ventricle with each contraction. Left ventricle systolic volume (LVSV) is the total volume of the blood available in left ventricle during systole. LVSV and the SV of the left ventricle are estimated for the same heart beat.

In the present invention, at the first stage there is a system that provides a representation of a functional relationship between the first area 102 and the cardiac parameter, which is the stroke volume (SV). The representation of the functional relationship might be represented as CP1=F(x). Then the cardiac parameter, which is the stroke volume (SV), is determined based on the first area 102 by using the representation of the functional relationship.

The same is true for the second area. It is required to have a representation of a functional relationship between the second area 110 and the corresponding cardiac parameter, which is the left ventricle systolic volume (LVSV). The representation of the functional relationship might be represented as CP2=F(y). Then the cardiac parameter, which is the left ventricle systolic volume (LVSV), is determined based on the second area 110 by using the representation of the functional relationship.

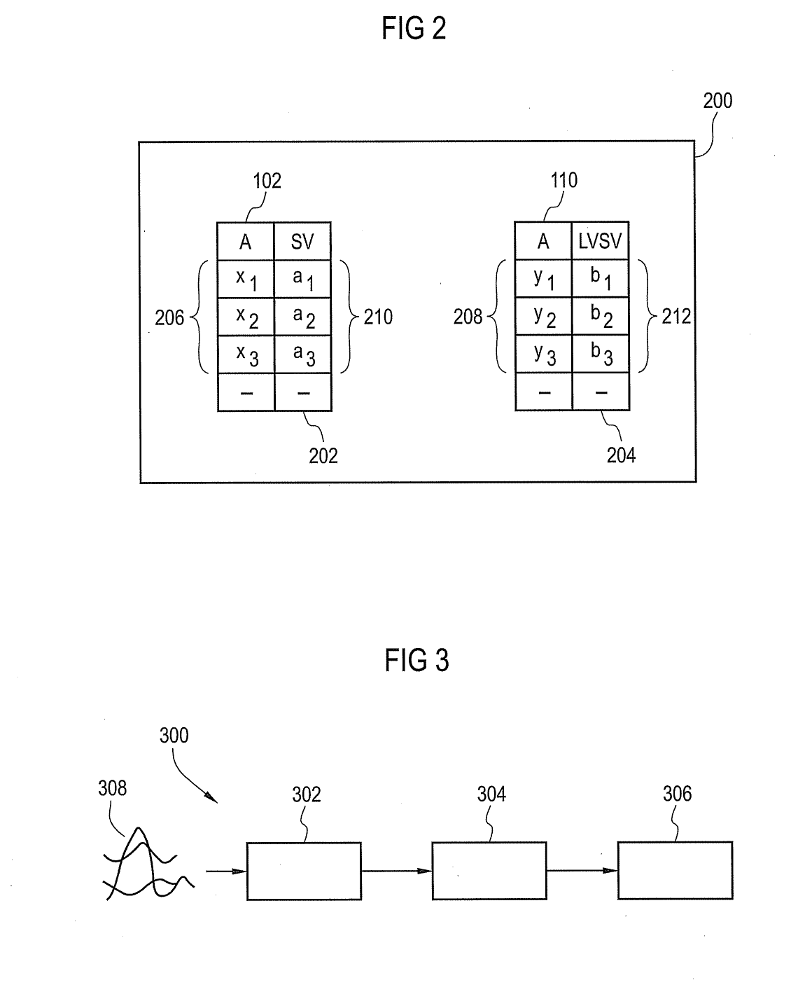

The representation of functional relationship can also be a database. For an example, FIG. 2 illustrates a database 200 which provide the functional relationship between the area under the curves and the corresponding cardiac parameters. The database 200 contains two tables 202 and 204. The plurality of entries 206, for example x1, x2, x3, . . . for the first area 102 is mapped to the corresponding cardiac parameters 210, for example a1, a2, a3 . . . measured using one or more of the conventional methods. Here the cardiac parameters 210, is the stroke volume (SV). Similarly, the plurality of entries 208, for example y1, y2, y3 . . . for the second area 110 is mapped to the corresponding cardiac parameter 212, for example b1, b2, b3 . . . measured using one or more of the conventional methods. Here the cardiac parameters 212, is the left ventricle systolic volume (LVSV). Practically, even a single table can be used for representing this kind of functional relationship. The representation of functional relationship can also be a function as discussed which maps the first area 102 and second area 110 to the corresponding cardiac parameter. The functional relationship is established by a series of measurements of the first area 102 and the second area 110 and the respective cardiac parameter independently.

The functional relationship is obtained by analyzing the pressure waveforms and estimating the area under the curves during systole of aortic wave fours and area under the curve during systole of left ventricle pressure waveform of known volumes like left ventricle systolic volume and stroke volume obtained in a large population involving both normal and diseased states using conventional methods like for example, left ventricle angiography.

The volumes obtained using this conventional method is then tagged with the corresponding value of area under the curve in the database. The values are standardized or arranged or filtered based on age, sex, heart rate and type of disease. The method used for calculating the area under curve during real time analysis would be the same method used for creating the database, which maps the area under curve and the corresponding cardiac parameter.

In the cathlab, the pressure waveforms are usually analyzed to obtain parameters like systolic, diastolic and mean blood pressure. Also by using area under curves, which represent the wave fauns during systole it is possible to calculate various volumes like left ventricle end systolic volume (ESV), left ventricle end diastolic volume (EDV), cardiac output (CO) and ejection fraction (EF). On the contrary, the other methods which uses waveforms for cardiac estimation calculates only stroke volume and cardiac output.

The present invention involves finding an additional cardiac parameter, left ventricle end systolic volume (ESV). ESV is the residual amount of blood in the ventricle after ejection. This is calculated by the equation ESV=LVSV−SV, wherein LVSV is left ventricle systolic volume and SV is stroke volume.

The present invention also involves finding an additional cardiac parameter, left ventricle end diastolic volume (EDV). EDV is the filled volume of the ventricle prior to contraction. This is calculated by the equation EDV=SV+ESV, wherein SV is the stroke volume and ESV is the left ventricle end systolic volume.

The present invention also involves finding an additional cardiac parameter, cardiac output (CO). Cardiac output is defined as the volume of blood ejected by the left ventricle into the aorta per minute. This is calculated by the equation CO=SV*HR, wherein SV is the stroke volume and HR is the heart rate.

Again another additional cardiac parameter, ejection fraction can be calculated. Ejection fraction is the fraction of blood in the left ventricle ejected per beat. This is calculated by the equation

$$EF = [ \frac{( EDV - ESV )}{EDV} ] * 100 ,$$

wherein EDV is left ventricle end diastolic volume and ESV is left ventricle end systolic volume.

Another measure of heart output is cardiac index (CI), which is the cardiac output per square meter of body surface area. If the body surface area, for a person is known, then the cardiac index (CI) is calculated by the equation

$$CI = \frac{CO}{BSA} ,$$

wherein CO is the cardiac output and BSA is the body surface area.

FIG. 3 illustrates a system 300 for measuring cardiac parameters according to an embodiment of the invention. The system has a determining module 302 adapted to receive the cardiac waveforms 308. The determining module 302 then determines a first area 102 and a second area 110, where the first area 102 extends between an onset of systole and the end of the systole and the second area extends between a waveform representing a left ventricle pressure and a waveform adapted to represent the left atrium pressure of the same systole. The system 300 also has a representation module 304, adapted to present a functional relationship between the first area 102 and the second area 110 on the one hand side and the cardiac parameters on the other hand side. Finally a cardiac parameter determining module 306 is used for determining the cardiac parameters based on the first area 102 and the second area 110 by using the representation of the functional relationship.

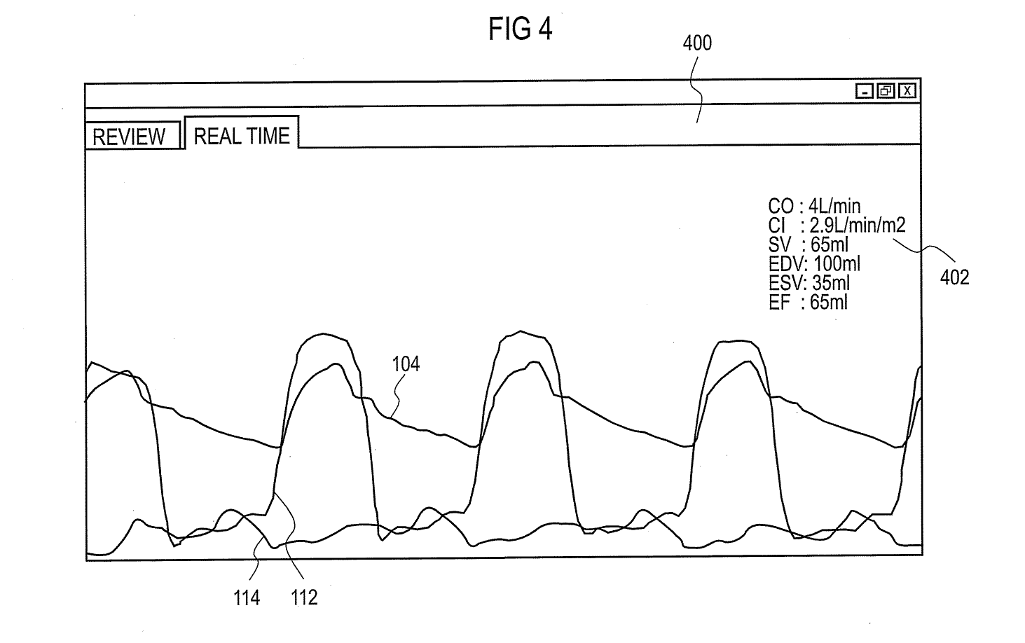

FIG. 4 illustrates the display 400, displaying pressure waveforms of aorta, left ventricle and pulmonary capillary wedge pressure (PCWP) obtained and displayed simultaneously on a common scale. Since the functional relationship is already available in the foam of a database or a function, it is always easy and fast to arrive at multiple cardiac parameter of a patient in real time. One way of representing the cardiac parameters of a patient is shown in FIG. 4. The figure represents a waveform 104 representing an aortic pressure, a waveform 112 representing a left ventricle pressure and a waveform 114 adapted to represent the left atrium pressure, which is the PCWP. Once the waves are displayed on a common scale, the system computes the required cardiac parameters as described earlier. The said computations and estimations of the area is performed the same way as explained earlier using the help of the database or the function. The physician can be provided with the results 402 of the cardiac parameters in the display unit as shown. Here the results read as, for example, CO: 4 L/min, CI: 2.9 L/min/m², SV:65 ml, EDV:100 ml, ESV:35 ml and EF:65%. It is to be noted that all the methodologies used here, to find the cardiac parameter of the left ventricle can be used for the right ventricle also.

Although the invention has been described with reference to specific embodiments, this description is not meant to be construed in a limiting sense. Various modifications of the disclosed embodiments, as well as alternate embodiments of the invention, will become apparent to persons skilled in the art upon reference to the description of the invention. It is therefore contemplated that such modifications can be made without departing from the embodiments of the present invention as defined.

1. A method for determining a cardiac parameter, comprising:
determining a first area under a waveform representing an aortic pressure, wherein the first area extends between an onset of a systole and an end of the systole;
providing a representation of a functional relationship between the first area and the cardiac parameter; and
determining the cardiac parameter based on the first area by using the representation of the functional relationship.

2. The method according to claim 1, further comprising:
determining a second area, wherein aid second area extends between a waveform representing a left ventricle pressure and a waveform adapted to represent a left atrium pressure in the systole;
providing the representation of the functional relationship between the first area and the second area and the cardiac parameter; and
determining the cardiac parameter based on the first area and the second area by using the representation of the functional relationship,
wherein the first area represents a stroke volume (SV) and the second area represents a left ventricle systolic volume (LVSV).

3. The method according to claim 1, wherein the representation of functional relationship is a database containing a plurality of entries for the first area and for the cardiac parameter.

4. The method according to claim 1, wherein the representation of functional relationship is a function which maps the first area to the cardiac parameter.

5. The method according to claim 1, wherein the representation of functional relationship is established by a series of measurements of the first area and the cardiac parameter independently.

6. The method according to claim 1, further comprises finding an additional cardiac parameter, left ventricle end systolic volume (ESV), calculated by the equation,
ESV=LVSV−SV
wherein, LVSV is left ventricle systolic volume and SV is stroke volume.

7. The method according to claim 1, further comprises finding an additional cardiac parameter, left ventricle end diastolic volume (EDV), calculated by the equation,
EDV=SV+ESV
wherein, SV is stroke volume and ESV is left ventricle end systolic volume.

8. The method according to claim 1, further comprises finding an additional cardiac parameter, cardiac output (CO), calculated by the equation,
CO=SV*HR
wherein SV is stroke volume (SV) X heart rate (HR).

9. The method according to claim 1, further comprises finding an additional cardiac parameter, ejection fraction, calculated by the equation,
$$EF = [ \frac{( EDV - ESV )}{EDV} ] * 100$$
wherein EDV is left ventricle end diastolic volume and ESV is left ventricle end systolic volume.

10. A method for determining a cardiac parameter, comprising:
determining a second area, wherein the second area extends between a waveform representing a left ventricle pressure and a waveform adapted to represent a left atrium pressure in a systole;
providing a representation of a functional relationship between the second area and the cardiac parameter; and
determining the cardiac parameter based on the second area by using the representation of the functional relationship.

11. The method according to claim 10, further comprising:
determining a first area under a waveform representing an aortic pressure, wherein the first area extends between an onset of a systole and an end of the systole;
providing the representation of the functional relationship between the first area and the second area and the cardiac parameter; and
determining the cardiac parameter based on the first area and the second area by using the representation of the functional relationship,
wherein the second area represents a left ventricle systolic volume (LVSV) and the first area represents a stroke volume (SV).

12. The method according to claim 10, wherein the representation of functional relationship is a database containing a plurality of entries for the second area and for the cardiac parameter.

13. The method according to claim 10, wherein the representation of functional relationship is a function which maps the second area to the cardiac parameter.

14. The method according to claim 10, wherein the representation of functional relationship is established by a series of measurements of the second area and the cardiac parameter independently.

15. A system for measuring a cardiac parameter, comprising:
a determining module adapted for determining a first area and a second area, wherein the first area is under a waveform in representing an aortic pressure and extends between an onset of systole and an end of the systole, and wherein the second area extends between a waveform representing a left ventricle pressure and a waveform adapted to represent a left atrium pressure of the systole;
a representation module adapted for presenting a functional relationship between the first area and the second area and the cardiac parameter; and
a cardiac parameter determining module for determining the cardiac parameter based on the first area and the second area by using the representation of the functional relationship.

16. The system according to claim 15, wherein the representation of functional relationship is a database containing a plurality of entries for the first area, the second area and the cardiac parameter.

17. The system according to claim 15, wherein the representation of functional relationship is a function which maps the first and second area to the corresponding cardiac parameter.

18. The system according to claim 15, wherein the first area represents a stroke volume (SV).

19. The system according to claim 15, wherein, the second area represents a left ventricle systolic volume (LVSV).
